(19) United States
(12) Patent Application Publication
(10) Pub. No.: US 2008/0210231 A1
(43) Pub. Date: Sep. 4, 2008

(54) METERED DOSE INHALER CLEANING METHOD AND APPARATUS

(75) Inventors: Prashant P. Kakade, Sunnyville, CA (US); Patrick O'Hara, Madison, NJ (US); Charles A. Buckner, Chapel Hill, NC (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/014,309

(22) Filed: Jan. 15, 2008

Related U.S. Application Data
(60) Provisional application No. 60/887,422, filed on Jan. 31, 2007.

(51) Int. Cl. A61M 11/00

(52) U.S. Cl. 128/200.23; 128/203.12

(57) ABSTRACT

A canister of pressurized medicament is removed from a metered dose inhaler actuation assembly and thereafter subjected to a cleaning firing to dislodge any material that can be built up on the interior of the canister's valve stem. During the cleaning firing, backpressure in the valve stem is reduced and/or eliminated by routing the resulting flow to atmosphere along an improved flow path. The improved flow path can be such that there are no constrictions less than about 75% of the size of the valve stem's outlet port. Thus, the backpressure experienced by the valve stem is greatly reduced versus that experienced during a normal dosing firing. As a result, the flow rate of medicament through the valve stem during the cleaning firing can be higher than during a normal dosing firing, allowing the built-up material to be dislodged during the cleaning firing.

### CROSS-REFERENCE SECTION TO RELATED APPLICATIONS

This application claims the benefit of U.S. Provisional Patent Application No. 60/887,422, filed Jan. 31, 2007, which is hereby incorporated by reference in its entirety.

### BACKGROUND

The present invention relates to metered dose inhalers for delivery of medicament to patients via aerosolization of the medicament, and related cleaning methods and devices.

Delivery of drugs via metered dose inhalers is well known for treating various conditions such as asthma. Typically, a metered dose inhaler includes a canister containing a pressurized supply of medicament that is mated to an inhaler actuator assembly. Typically, the supply of medicament is triggered by displacing a hollow valve stem of the canister toward the main body of the canister. This releases a metered portion of the medicament (e.g., a predetermined metered amount) into the inhaler actuator assembly. The significant pressure differential between the canister pressure and the atmosphere results in the released material being propelled through the inhaler actuator assembly. More particularly, the released material is typically directed through a very small nozzle orifice (or “nozzle”) that aerosolizes the spray. This aerosolized spray is then inhaled by the patient so as to be preferentially deposited in the lungs.

The nozzle orifice is an important determinant of the resultant aerosol droplet size and size distribution. A smaller orifice tends to produce a finer spray, which is typically more therapeutically effective. However, a small nozzle orifice also creates a constriction in the flow path, which creates some backpressure that is communicated along the valve stem. The presence of this backpressure causes the flow of material through the valve stem to be slower than it otherwise would be. Because of the slower flow rate, there is an increased possibility of having some medicament deposit on the interior of the valve stem. Build up of the medicament inside the valve stem can undesirably lead to sporadic break off of the material, which can result in clogging of the downstream nozzle orifice, or lead to undesirable biological contamination of the device, or affect dose uniformity.

The extent of deposition inside the valve stem depends on the medicament formulation, valve stem material properties, and on the design of the inhaler actuator assembly. Conventional metered dose inhalers for asthma may not experience significant build up of material in the valve stem due to the formulations used. Thus, while patients arc typically instructed to clean the inhaler using various techniques, no known cleaning approach addresses cleaning of the inside of the canister valve stem. However, as metered dose inhalers are adapted for use with other formulations, such as formulations with larger molecule sizes (e.g., macromolecules) and/or different chemistries, internal valve stem build up is believed to become more of a consideration for proper operation.

Thus, there remains a need for alternative approaches to using and/or cleaning metered dose inhalers, advantageously approaches that allow the interior of the valve stein to be easily cleaned.

### SUMMARY OF THE INVENTION

Various embodiments of the present invention are intended to allow a canister of pressurized medicament to be removed, fully or partially, from a metered dose inhaler actuation assembly and thereafter subjected to a cleaning actuation to dislodge undesirable material that can be present on the interior of the canister's valve stem. During the cleaning actuation, backpressure in the valve stem is substantially reduced and/or eliminated by routing the resulting flow to atmosphere along an improved flow path. The improved flow path in some embodiments, has no constrictions less than about 75% of the size of the valve stem's outlet port, and advantageously no constrictions less than the size of the valve stem's outlet port. Thus, the backpressure experienced by the valve stem is greatly reduced versus that experienced during a normal dosing actuation. As a result, the flow rate of medicament through the valve stem during the cleaning actuation can be higher than during a normal dosing actuation, allowing the built-up material to be dislodged during the cleaning actuation.

In one embodiment, a method of cleaning at least a portion of a metered dose inhaler comprises: joining a canister to a body; the canister having a pressurized supply of medicament therein and a hollow outlet stem terminating at an outlet port; the canister operative to output a metered amount of the medicament via the outlet port in response to being triggered: the outlet port having a first size; the body having a passage therethrough; triggering the canister to fire by displacing the canister toward the body passage so as to thereby spray medicament from the canister into the passage to generate a pressurized exhaust from the passage; routing the exhaust from the passage to atmosphere such that the exhaust encounters no constrictions smaller than about 75% of the first size. The exhaust can be routed directly to atmosphere or can be routed through a collection chamber associated with the body.

Other aspects of various embodiments of a related inventive device and other related methods are also disclosed in the following description. The various aspects can be used alone or in any combination, as is desired.

### BRIEF DESCRIPTION OF THE DRAWINGS

FIG. 1 shows a metered dose inhaler of the prior art.

FIG. 1A shows an enlarged partial cut-away view of the canister of FIG. 1.

FIG. 2 shows a canister and a cleaning body according to one embodiment of the present invention.

FIG. 3A shows a cross-section taken along line III-III with the canister in the ready position.

FIG. 3B shows a cross-section taken along line III-III with the canister in the firing position.

FIG. 3C shows an enlarged view of a portion of FIG. 3A.

FIG. 4 shows a canister and a cleaning body according to another embodiment of the present invention.

FIG. 5A shows a cross-section of the cleaning body along line V-V.

FIG. 5B shows the assembly of FIG. 4 with the cleaning body cross-sectioned at line V-V.

FIG. 6 shows a canister and a cleaning body according to another embodiment of the present invention with an extended flange to actuate a dose counter associated with the canister.

FIG. 7 shows the canister and cleaning body of FIG. 4 being inserted into an inhaler assembly in preparation for a cleaning actuation.

### DETAILED DESCRIPTION OF THE INVENTION

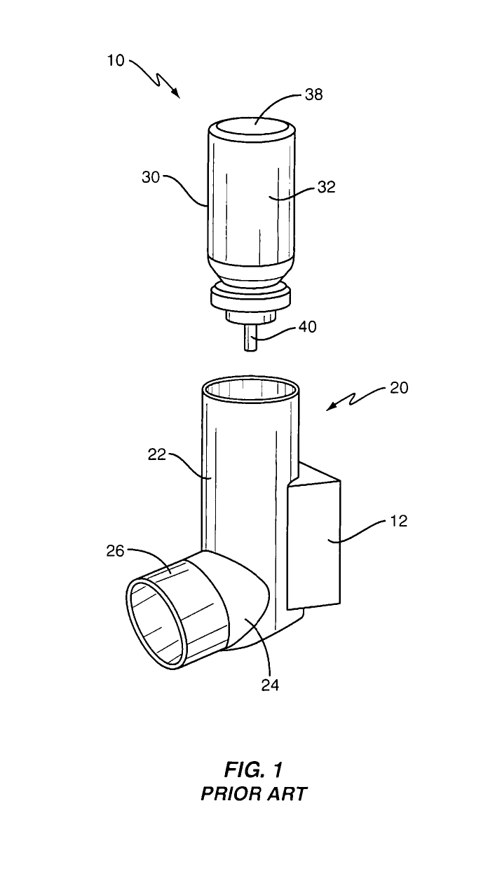

The present invention relates to metered dose inhalers. Referring to FIG. 1 and FIG. 1A, a metered dose inhaler 10 typically includes a canister 30 and an inhaler assembly 20. The canister 30 typically includes a main canister body 32 and a valve stem 40 moveably coupled thereto. The main canister body 32 advantageously takes the generally cylindrical form shown in FIG. 1. The operative end of the canister body 32, sometimes referred to as the canister ferrule, typically includes a generally cylindrical central boss 36 peripherally surrounded by an annular collar 34 of larger diameter. The canister body 32 houses a medicament 5 stored under pressure, typically due to the presence of a low vapor pressure propellant, and therefore acts as a pressure vessel. The valve stem 40 extends forward from the main canister body 32, and typically takes the form of a hollow cylindrical tube with an internal bore 44 of relatively constant internal diameter. The valve stem 40 typically includes a small radially oriented hole (not shown) towards its proximate end that acts as an input to the internal bore 44 in a fashion well known in the art. The distal end of the valve stem 40 forms the valve stem outlet port 46, and typically takes the form of a simple circular opening of a known size X. The valve stem 40 is moveably mated to the main canister body 32 so as to be displaceable between a ready position relatively away from the main body 32 and a firing position relatively closer to the main body 32, and is biased toward the ready position. Movement of the valve stem 40 toward the main canister body 32, from the ready position to the firing position, allows the pressurized medicament to enter the central bore 44 of the valve stem. The pressurized medicament rapidly expands to fill the central bore 44 and flows out outlet port 46 as a pressurized spray.

The inhaler assembly 20 can take a wide variety of forms known in the art. One exemplary inhaler assembly 20 is a generally L-shaped body as shown in FIG. 1. The lower portion 24 of the “L” includes a mouthpiece 26 from which the medicament is drawn and/or propelled into the patient's mouth. The upper portion 22 of the “L” includes a recess for receiving the canister 30. The inhaler assembly 20 includes suitable internal structures to allow the canister 30 to be triggered for the delivery of one “charge” of medicament in any fashion known in the art, so that the medicament can be administered to a patient by an inhalation technique. Further, the inhaler assembly 20 can include a variety of features, including a dose counter 12, a vortex nozzle, breath actuation mechanisms, and the like, as is known in the art. Attention is directed to U.S. Pat. No. 6,418,925: and to U.S. patent applications Ser. No. 10/625,359 (U.S. Patent Application Publication No. 2005/0028815) and Ser. No. 10/908,133 (U.S. Patent Application Publication No. 2006/0243275), which are all incorporated herein by reference.

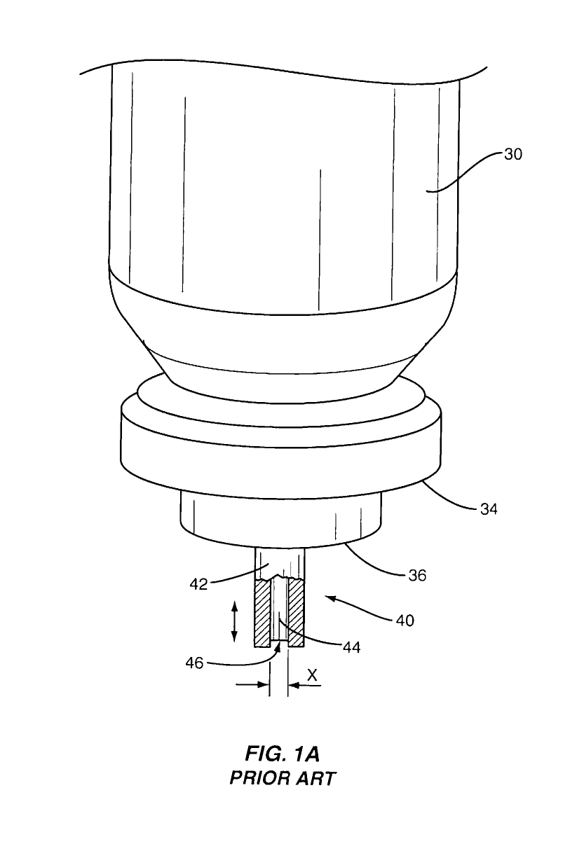

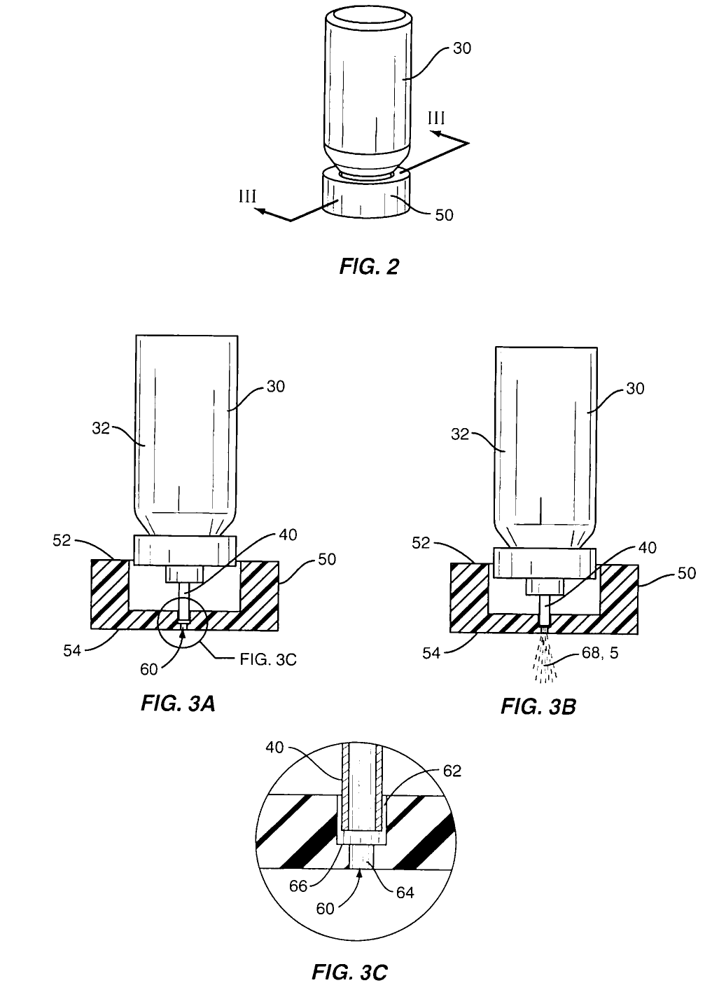

In exemplary embodiments of the present invention, the canister 30 is removed from inhaler assembly 20 after several firings, joined to a separate cleaning body 50 for cleaning of the interior of valve stem 40, and then rejoined to the inhaler assembly 20. One embodiment of a cleaning body 50 is shown in FIG. 2-3C. The cleaning body 50 of FIG. 2 is generally disc-shaped and advantageously significantly larger in diameter than the canister main body 32. A central bore or passage 60 leads from the upper surface 52 to the lower surface 54 of cleaning body 50. The central passage 60 has a proximal upper portion 62 of a relatively larger diameter and a distal lower portion 64 of a relatively smaller diameter that are advantageously collinear. The proximal portion 62 can advantageously be of a constant diameter, but can include a tapered entry if desired. The proximal portion 62 is sized and shaped to receive the valve stem 40, and therefore can advantageously have a diameter just slightly larger than the exterior of the valve stern 40. In contrast, the distal portion of the passage is advantageously sized to be smaller than the exterior of the valve stem 40, but larger than the bore 44 of valve stem 40. The distal portion 64 can likewise be of a constant diameter. The two sections 62,64 interface in abutting fashion to form an internal shoulder 66. Thus, the outlet port 46 of valve stem 40, when the valve stem 40 abuts shoulder 66, opens directly into passage distal portion 64.

The medicament can be administered to the patient by initially mating the canister 30 to the inhaler assembly 20 in any conventional fashion. The canister 30 is then triggered to supply the medicament 5 to the aerosolizing portions of the assembly 20, and the aerosolized medicament is inhaled by the patient. A single “charge” of medicament 5 can correspond to a dose, or a dose can comprise multiple charges taken in short succession. This dosing regimen is advantageously repeated multiple times over a period of time. For example, the dosing regimen can be repeated three times a day for several days. When the dosing regimen exceeds a threshold, the valve stem 40 is cleaned. This threshold can be set as a given number of actuations of the canister 30, or a given amount of time, or a combination thereof. Or, a cleaning cycle can be initiated if the patient suspects some problem with the inhaler 10.

To clean the valve stem 40, the canister 30 is removed from the inhaler assembly 20 and joined to the cleaning body 50. To do this, the valve stem 40 is inserted into the upper portion 62 of cleaning body central passage 60 until the valve stem 40 abuts against shoulder 66. The canister 30 is pressed toward the cleaning body 50, so that the valve stem 40 presses against shoulder 66. During this action, the combination of the cleaning body 50 and the canister 30 can be supported in a variety of ways. For example, the lower surface 54 of cleaning body 50 can be supported by two spread fingers of one hand and the thumb of the hand placed over the flat base 38 of canister 30 (which is facing away from cleaning body 50). As the canister 30 is pressed, the valve stem 40 is forced to move relative to the canister main body 32, until the valve stem 40 relatively retracts to the firing position.

Movement of the valve stem 40 to the firing position causes a charge of pressurized medicament to be released into the valve stem 40, where it rapidly expands and flows out stem outlet port 46 due to the relative pressure differential. The medicament 5 flows directly from the stem outlet port 46 into lower portion 64 of passage 60, though the lower portion 64, and out the passage 60 as an exhaust 68. As can be appreciated, this exhaust 68 is vented directly to atmosphere in this embodiment because passage 60 opens directly to atmosphere. As Such, exhaust 68 does not encounter any constrictions less than the size of valve stem outlet port 46 before being reaching ambient atmosphere. Because of this, it is believed that that significant backpressure is not generated in the valve stem 40, and the resulting flow rate through the valve stem 40 is maximized. It should be noted that this flow rate is higher than that experienced during normal inhalation activation. The higher flow rate tends to strip away any undesired deposits that could have formed inside bore 44 of valve stem 40. Further, any stripped material is simply carried away to atmosphere in exhaust 68, and therefore cannot result in blockage of the nozzle orifice of the inhaler assembly 20. In most situations, only one firing of the canister 30 will be appropriate for complete cleaning, although multiple firings can be appropriate in some situations. After the cleaning firing(s), the canister 30 is removed from the cleaning body 50 and re-mated to the inhaler assembly 20. If desired, the exterior 42 of valve stem 40 can be striped or otherwise cleaned prior to re-mating with the inhaler assembly 20. Also, the inhaler assembly 20 can be cleaned using any appropriate technique while the canister 30 is unmated therefrom. The inhaler 10 is then ready for additional use.

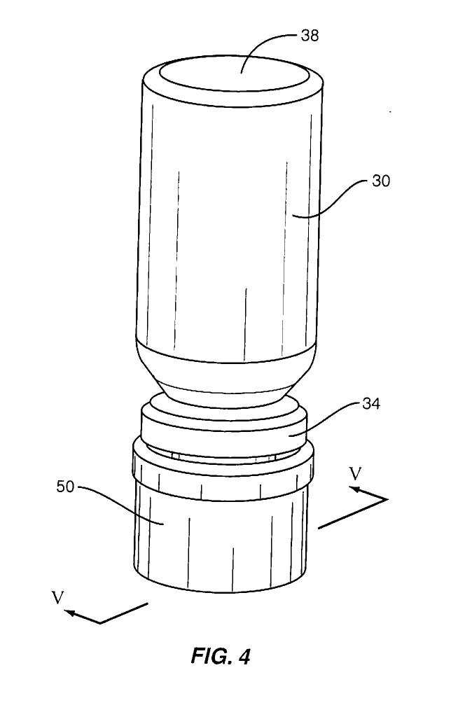

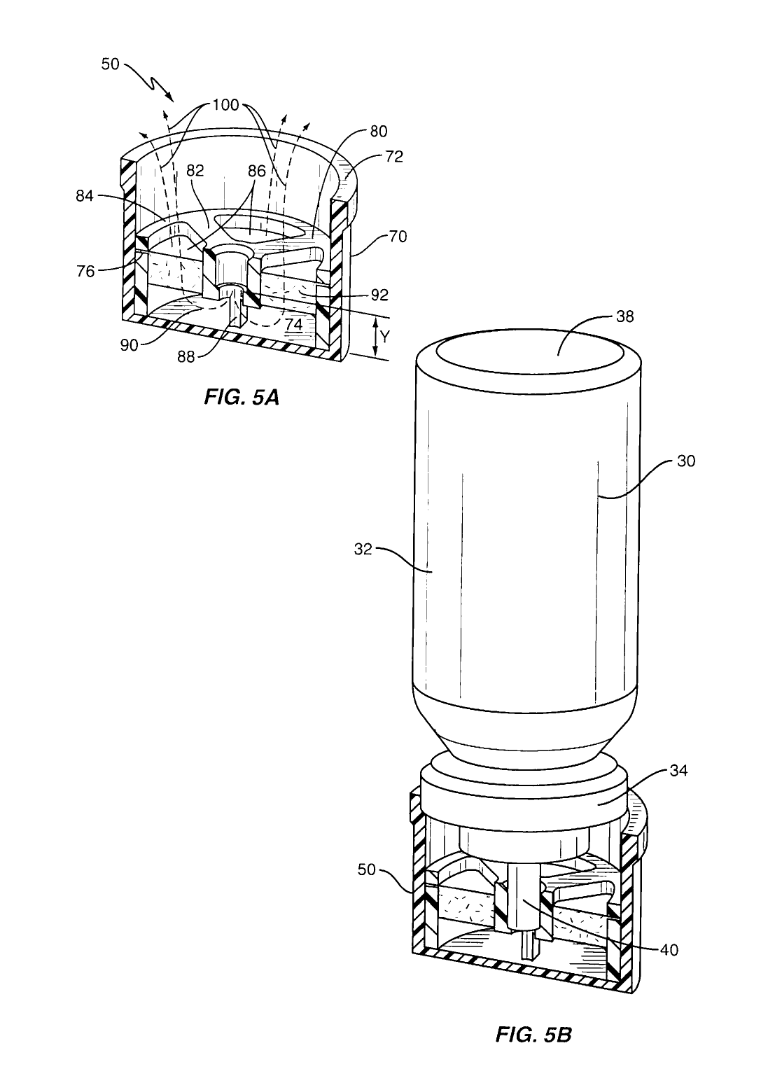

In other embodiments, the cleaning body 50 can be more complex. For example, a more complex cleaning body 50 is shown in FIGS. 4-5B. This cleaning body 50 includes an outer housing 70 and a stop plate 80. The housing 70 is a generally cylindrical hollow body, with an open proximal end and a closed distal end. The proximal end of the housing includes a peripheral rim 72, but is otherwise open. The distal end of the housing is bounded by an impingement surface 74 disposed generally normal to the axis of housing 70. An interior shelf 76 runs around the interior periphery in spaced relation to impingement surface 74. The stop plate 80 in this example is a relatively thin, generally circular, body with a central passage 60 and a plurality of exhaust outlets 86. As with the cleaning body 50 of FIG. 2, the central passage 60 in FIGS. 5A-5B includes a proximal portion 62 of a relatively larger diameter and a distal portion 64 of a relatively smaller diameter. The proximal portion 62 and distal portion 64 are advantageously collinear and of respective constant diameters, and the two sections 62,64 interface in abutting fashion to form an internal shoulder 66. The proximal portion 62 is advantageously sized and shaped to just receive valve stem 40, and therefore has a diameter just slightly larger than the exterior 42 of valve stem 40. The distal portion 64 of passage 60 is advantageously sized to be smaller than the exterior 42 of valve stem 40, but larger than bore 44 of valve stem 40. Thus, the interior of the valve stem 40 opens directly into the distal portion 64 of passage 60 at outlet port 46. The shoulder 66 is advantageously spaced from the impingement surface 74 by a distance Y that is approximately the same as the length of valve stem 40 or more. The exhaust outlets 86 can be defined by a plurality of spokes 82 that extend generally radially from proximate central passage 60 laterally toward peripheral rim 84 of stop plate 80. The combined cross-sectional area of the exhaust outlets 86 is advantageously larger than the cross-sectional area of valve stem outlet port 46; indeed, the cross-sectional area of each individual exhaust outlet 86 is advantageously larger than the cross-sectional area of valve stem outlet port 46. The peripheral rim 84 rests against shelf 76 so that the main portion of stop plate 80 is held in spaced relation from impingement surface 74. If desired, stop plate 80 can also include one or more distally extending support posts 88 for aiding in support of stop plate 80 in this position. As can be seen, a collection chamber 90 is formed between the distal surface of stop plate 80 and impingement surface 74. The central passage 60 acts as an inlet to this chamber 90, and the exhaust outlets 86 act as the outlet for this chamber 90. Upon firing of the canister 30, the exhaust 68 from the central passage 60 flows along a flow path 100 from the central passage 60 into chamber 90, through the chamber 90, and out to ambient atmosphere via exhaust outlets 86. Note that for optimal performance, the canister annular collar 34 should be spaced from the proximal peripheral rim 72 of the cleaning body 50, when the valve stem 40 is in the firing position, by an amount such that the cross-sectional area between the two is larger than the cross-sectional area of valve stem outlet port 46. As such, the exhaust 68 again does not encounter any constrictions less than the size of the valve stem interior cross-section along flow path 100 before being reaching ambient atmosphere.

The cleaning body 50 embodiment of FIG. 5 can be used similarly to the embodiment of FIG. 2. However, the cleaning body 50 of FIG. 5 can be more easily held between a single finger and a thumb, and the ultimate gaseous output of the cleaning process is directed more radially than longitudinally. Further, it should be noted that exhaust 68 from passage 60 is directed at impingement surface 74 rather than at the surroundings. Further still, some embodiments can include an optional absorbent material (e.g., foam) 92 disposed just upstream of the exhaust outlets 68. This absorbent material 92 can help absorb medicament 5 that is sprayed into chamber 90.

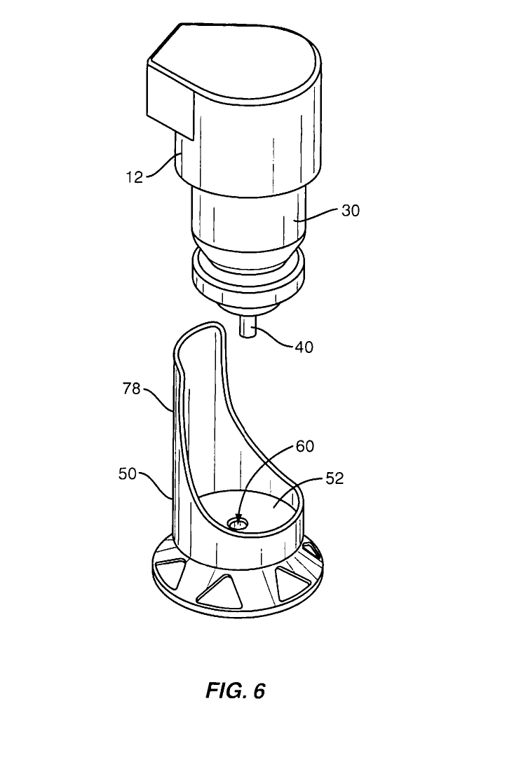

It has been assumed above that cleaning body 50 does not substantially longitudinally overlap canister main body 32. However, in some embodiments, a portion of the cleaning body 50 can longitudinally overlap a significant length of the canister main body 32. For example, the cleaning body 50 of FIG. 6 includes a proximal flange portion 78 of substantial longitudinal length. This flange 78 can advantageously be curved so as to follow the cylindrical side of the canister main body 32, but with a larger radius of curvature so that a gap is formed therebetween. The purpose of the flange 78 is to actuate an optional dose counter 12 that can be associated with the canister 30. Thus, the flange 78 should be of sufficient length so as to be able to reach the relevant portions of dose counter 12. In other embodiments, the flange 78 can be extended so as to substantially or fully peripherally enclose canister 30, as can be appropriate. The flow path 100 of the exhaust 68 for such a cleaning body 50 can be similar to any of those discussed above, and care should be taken with such embodiments to have adequate clearances and/or dedicated openings to avoid creating undesirable constrictions in the flow path 100.

The discussion above has assumed that the flow path 100 from the valve stem outlet port 46 reaches ambient atmosphere without encountering any constrictions smaller than the cross-sectional size of the valve stem interior. However, slightly smaller constrictions can be present in some embodiments of the present invention. For example, the lower portion 64 of passage 60 call have a cross-section that is 75% of the cross-sectional size of the valve stem interior. Advantageously, this size is larger, such as 80%, 85%. 90%, or 95%, with larger size ratios being preferred. It is believed that a size ratio of ≧100% is more advantageous, but constrictions sized between 100% and about 75% (inclusive) cannot generate significant backpressure, and the flow rates of the medicament through the valve stem 40 can be maintained at desired levels. These sizes are in stark contrast to the typical 1%-5% sizes of the nozzle orifices typically employed.

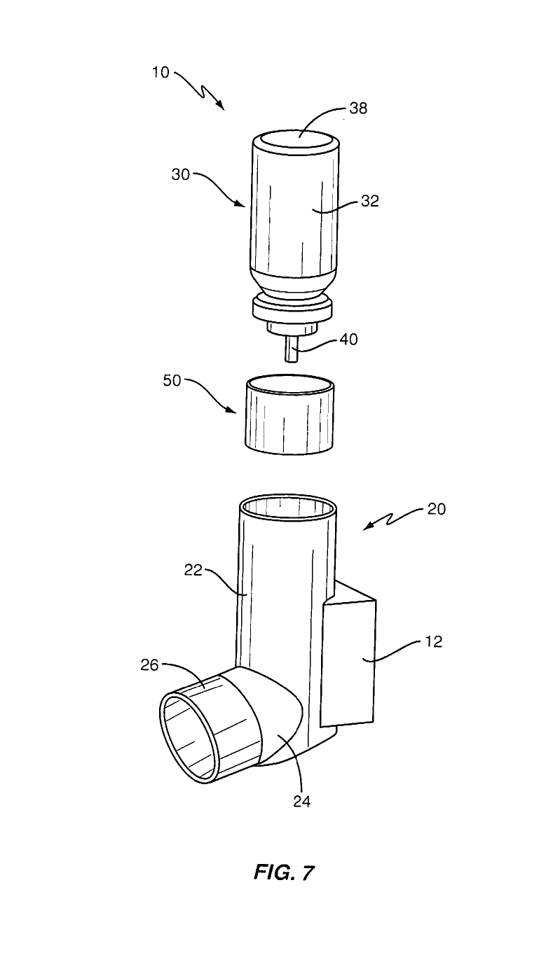

The discussion above has assumed that the cleaning body 50 is directly supported by the user's hand; however, such is not required in all embodiments. In some embodiments, the cleaning body 50 can be supported by a suitable fixture, which in turn can be handheld or placed on a suitable surface during the cleaning actuation. Indeed, as shown in FIG. 7, the cleaning body 50 can be inserted into the inhaler assembly 20 for the cleaning operation, and subsequently removed. Further still, the cleaning body 50 can be formed as a suitable exterior feature (not shown) on the inhaler assembly 20.

Several tests have been run to examine the effect of using a cleaning body 50, and the results indicate that following a cleaning regimen that encourages periodic cleaning of the valve stem interior will lead to better performance. Each of the tests used a pressurized insulin medicament in the canister 30, with the formulation being a relatively high strength suspension formulation. The weight of the medicament sprayed from the canister (“shot weight”) was measured for each actuation, with a ten second wait between each actuation. A substantially similar inhaler assembly 20 was used for each test, and three canisters were tested at each test condition.

Test A used a five minute wait between sets of three actuations, without a cleaning regimen. The inhaler was found to be clogged after approximately twenty-seven actuations of the first canister. The second canister caused clogging after approximately forty-five actuations. The third canister did not cause clogging after one hundred twenty actuations, but showed a marked variation in shot weight around sixty actuations and again around eighty actuations.

Test B used a sixty minute wait between sets of three actuations, without a cleaning regimen. The inhaler was found to be clogged after approximately thirty-six actuations of the first canister. The other two canisters caused clogging after approximately eighty-seven actuations each.

Test C used a five minute wait between sets of three actuations (similar to Test A), with a cleaning actuation using a cleaning button 50 after every thirty actuations. The inhaler was found to be clogged after approximately ninety-two actuations for the first canister. The second canister caused clogging after approximately one hundred eighteen actuations. The other canister did not clog or show a significant drop off in shot weight until the canister was emptied after approximately one hundred forty actuations.

Test D was similar to Test C, but the cleaning cycle was changed to be a cleaning actuation using a cleaning button 50 after every twenty actuations. None of the three canisters caused clogging or showed a significant drop off in shot weight through approximately one hundred forty actuations (at which point the canisters were emptied).

The instances of clogging during the testing outlined above appear to be the result of build up of material on the inside of the valve stem, and subsequent break off of the agglomerated material. It is believed that the broken off material moves downstream, probably during that “shot” but possibly during a subsequent shot, to block a downstream constriction, such as the nozzle orifice.

Based on the above, it is clear that subjecting the canisters 30 to a cleaning regimen using a cleaning button can improve performance of an inhaler 10. It is believed that the appropriate frequency of the cleaning regimen will vary based on a number of factors, including medicament composition, valve stem material, valve stem size, nozzle orifice size, and the like. Nevertheless, a cleaning frequency of approximately every ten to twenty actuations is believed appropriate for most situations.

The present invention can be carried out in other specific ways than those herein set forth without departing from the scope and essential characteristics of the invention. Further, the various aspects of the disclosed device and method can be used alone or in any combination, as is desired. The disclosed embodiments are, therefore, to be considered in all respects as illustrative and not restrictive, and all changes coming within the meaning and equivalency range of the appended claims are intended to be embraced therein.

What is claimed is:

1. A method of cleaning at least a portion of a metered dose inhaler, comprising:
joining a canister to a body;
the canister having a pressurized supply of medicament therein and a hollow outlet stem terminating at an outlet port; said canister operative to output a metered amount of said medicament via said outlet port in response to being triggered; said outlet port having a first size;
the body having a passage therethrough;
triggering the canister to fire by displacing the canister toward the body passage so as to thereby spray medicament from the canister into the passage to generate a pressurized exhaust from the passage;
routing the exhaust from the passage to atmosphere along a flow path such that the exhaust encounters no constrictions that result in said flow path having a size smaller than about 75% of the first size.

2. The method of claim 1 wherein said routing comprises routing the exhaust directly from the passage to atmosphere.

3. The method of claim 1 wherein said routing comprises routing the exhaust through a chamber associated with the body.

4. The method of claim 3 wherein an absorbent material is disposed along a flow path of the exhaust downstream of the passage and upstream of atmosphere;
and further comprising absorbing at least a portion of said medicament with said absorbent material as the exhaust passes through the chamber.

5. The method of claim 1 wherein said passage is substantially straight throughout.

6. The method of claim 1 further comprising joining the canister to an inhaler actuator assembly, and thereafter removing the canister from the inhaler actuator assembly, both prior to said joining the canister to the body.

7. The method of claim 1 further comprising firing said canister a plurality of times prior to said joining the canister to the body.

8. The method of claim 1 wherein the exhaust exits the passage in a first direction, and further comprising impinging the exhaust against a surface disposed generally transverse to the first direction and spaced from the outlet port.

9. The method of claim 1 wherein routing the exhaust from the passage to atmosphere comprises routing the exhaust from the passage to atmosphere such that the exhaust encounters no constrictions smaller than about 85% of the first size.

10. The method of claim 1 wherein routing the exhaust from the passage to atmosphere comprises routing the exhaust from the passage to atmosphere such that the exhaust encounters no constrictions smaller than about the first size.

11. A method of cleaning at least a portion of a metered dose inhaler, comprising:
joining a canister to a body;
the canister having a pressurized supply of medicament therein and a hollow outlet stem terminating at an outlet port; said canister operative to output a metered amount of said medicament via said outlet port in response to being triggered; said outlet port having a first cross-sectional area;
the body having a passage therethrough; said passage having a first section abutting a second section at a shoulder; the first section having a shape generally corresponding to an exterior of said stem; said second section having second cross-sectional area throughout its length of 75% or more of said first cross-sectional area of the stem outlet port;
wherein joining the canister to the body comprises disposing the outlet stem in the passage first section;
thereafter, pressing the outlet stem against the shoulder so as to thereby spray an output from the canister through said second section of the passage;
wherein a flow path for the canister output, starting immediately after the passage and ending at atmosphere, includes no constrictions smaller than about 75% of said first cross-sectional area of the stem outlet port.

12. The method of claim 11 wherein said second cross-sectional area is not less than approximately said first cross-sectional area.

13. The method of claim 12 wherein said second cross-sectional area is greater than said first cross-sectional area.

14. The method of claim 11 wherein said body passage is substantially collinear with said stem.

15. The method of claim 11 wherein said passage is substantially straight throughout.

16. The method of claim 11 wherein said passage is open to atmospheric pressure at an end thereof opposite said stem.

17. The method of claim 11 further comprising joining the canister to an inhaler actuator assembly prior to said joining the canister to the body.

18. The method of claim 17 further comprising joining the canister to the inhaler actuator assembly after said pressing the outlet stem against the shoulder.

19. The method of claim 17 further comprising firing said canister a plurality of times prior to said disposing the outlet stem in the passage first section.

20. The method of claim 11 wherein said stem has an operative volume terminating at said outlet port; and wherein said second passage opens directly to an exhaust space having a volume larger than said stem operative volume.

21. The method of claim 20 wherein said body defines said exhaust space.

22. The method of claim 20 wherein said exhaust space is ambient atmosphere.

23. The method of claim 11 wherein spray passing through said second section of the passage exits said second section in a first direction, and further comprising impinging said spray against a surface disposed generally transverse to said first direction and spaced from said canister outlet port.

24. A method of cleaning at least a portion of a metered dose inhaler, comprising:
joining a canister to a body;
the canister having a pressurized supply of medicament therein and a hollow outlet stem terminating at an outlet port; said canister operative to output a metered amount of said medicament via said outlet port in response to being triggered; said outlet port having a first size;
the body having a passage therethrough;
triggering the canister to fire by displacing the canister toward the body passage so as to thereby spray medicament from the canister into the passage to generate a pressurized exhaust from the passage;
routing the exhaust from the passage to atmosphere along a flow path such that the flow path has an effective size of about 75% of the first size or more throughout.

25. The method of claim 24 wherein the flow path has an effective size of about the first size or more throughout.

26. A method of cleaning at least a portion of a metered dose inhaler, comprising:
providing a canister having a pressurized supply of medicament therein and a hollow outlet stem terminating at an outlet port; the canister operative to output a metered amount of the medicament under pressure via the outlet port in response to being triggered; the outlet port having a first cross-sectional area;
providing a body having a chamber and an inlet passage joining to the chamber; the inlet passage having a first upstream section abutting a second downstream section at an internal shoulder;
pressing the outlet stem against the shoulder so as to thereby spray medicament from the canister through the second section of the inlet passage and into the chamber;
exhausting the chamber at one or more outlets having a combined cross-sectional area that is not less than about 75% of said first cross-sectional area.

27. The method of claim 26 wherein an absorbent material is disposed in said chamber upstream from said outlets; and further comprising absorbing at least a portion of said medicament with said absorbent material.

28. The method of claim 26 wherein said combined cross-sectional area of said outlets is about said first cross-sectional area or more.

29. The method of claim 26 further comprising joining the canister to an inhaler actuator assembly and firing said canister a plurality of times prior to said pressing the outlet stem against the shoulder.

30. The method of claim 29 further comprising rejoining the canister to the inhaler actuator assembly after said exhausting.

31. An assembly comprising:
a canister having a pressurized supply of medicament therein and a hollow outlet stem terminating at an outlet port; said canister operative to output a metered amount of said medicament via said outlet port in response to being triggered; said outlet port having a first cross-sectional area;
a body having a passage therethrough; said passage having a first section abutting a second section at an internal shoulder; the first section having shape generally corresponding to an exterior of said stem; said second section having second cross-sectional area throughout its length of about 75% or more of said first cross-sectional area;
the outlet stem removably disposed in the passage first section and abutting the internal shoulder so that a spray output from the canister is directed through said second section of the passage;
a flow path extending from the outlet port through the body to atmosphere; wherein said flow path has cross-sectional area throughout of at least about 75% of said first cross-sectional area.

32. The assembly of claim 31 wherein said second cross-sectional area is not less than approximately said first cross-sectional area.

33. The assembly of claim 32 wherein said second cross-sectional area is approximately said first cross-sectional area.

34. The assembly of claim 32 wherein said second cross-sectional area is greater than said first cross-sectional area.

35. The assembly of claim 31:
wherein the stem is displaceable generally along its longitudinal axis from a first ready position to a second firing position;
wherein the canister further comprises a shoulder spaced from the outlet port by a first distance when the canister is disposed in the second firing position;
wherein the body further comprises an abutment face disposed proximate the canister and spaced from the passage internal shoulder by approximately the first distance.

36. The assembly of claim 31 wherein the body further comprises a chamber disposed along said flow path downstream from said passage.

37. The assembly of claim 36 further comprising
one or more outlets disposed downstream from said chamber; and
an absorbent material disposed in said chamber upstream from said outlets.

38. The assembly of claim 31 further comprising an inhaler actuator assembly adapted to receive said canister.

39. A method of administering a formulation to a human patient by inhalation, comprising:
(a) operatively connecting a canister to an inhaler actuator assembly; the canister comprising a medicament stored under pressure and a hollow outlet stem terminating at an outlet port: said outlet port having a first size;
(b) aerosolizing the medicament via the inhaler actuator assembly;
(c) inhaling the aerosolized medicament;
(d) repeating steps (b) and (c) a plurality of times over a period of time;
(e) after step (d), operatively disconnecting the canister from the inhaler actuator assembly and joining the canister to a cleaning body having a passage therethough;
(f) after step (e), triggering the canister to fire by displacing the canister toward the cleaning body passage so as to thereby spray medicament from the canister into the passage to generate a pressurized exhaust from the passage; said exhaust being routed along a flow path extending from the passage to atmosphere; the flow path having an effective size of about 75% of the first size or more throughout;
(g) after step (f), operatively disconnecting the canister from the cleaning body and operatively reconnecting the canister to the inhaler actuator assembly;
(h) repeating at least step (d).

40. The method of claim 39 wherein step (h) comprises repeating steps (d)-(g).

41. The method of claim 39 wherein said period of time is weekly.

42. The method of claim 39 wherein said plurality of times in step (d) is at least ten times.

43. The method of claim 39 wherein said inhaler actuator assembly and said cleaning body are separate and distinct from one another.

44. The method of claim 39 wherein the inhaler actuator assembly comprises a vortex nozzle: and wherein step (b) comprises aerosolizing the medicament via the vortex nozzle.

45. The method of claim 39 further comprising cleaning an exterior of said inhaler actuator assembly.

46. The method of claim 45 wherein said cleaning an exterior of said inhaler actuator assembly occurs after said operatively disconnecting the canister from the inhaler actuator assembly and prior to said operatively reconnecting the canister to the inhaler actuator assembly.
